(19) United States
(12) Patent Application Publication
(10) Pub. No.: US 2025/0345528 A1
(43) Pub. Date: Nov. 13, 2025

(54) SAFETY DEVICE FOR A SYRINGE

(71) Applicant: Alfredo RICCI, Pescara (IT)

(72) Inventor: Alfredo RICCI, Pescara (IT)

(21) Appl. No.: 18/862,631

(22) PCT Filed: Apr. 27, 2023
(86) PCT No.: PCT/IB2023/054349

§ 371 Date: Nov. 4, 2024

(30) Foreign Application Priority Data
May 4, 2022 (IT) 102022000009104

(51) Int. Cl. A61M 5/32

(52) U.S. Cl. CPC A61M 5/3204; A61M 5/3232; A61M 2205/19

(57) ABSTRACT

A safety device for a syringe including a connector which can be coupled to a syringe, a main body provided with a cavity and which can be coupled to the connector so as to extend above it and a needle body for supporting and housing a needle or cannula, the needle body longitudinally extending in the connector and in the main body. The device further includes a push element which can be coupled to a distal end of the needle body and movable inside the connector by action of an axial thrust of a plunger of the syringe, and a needle support which can be coupled to the needle body and is operable by the push element between a locked position of the needle body, wherein the needle support is interposed between the connector and the needle body, and an unlocked position of the needle body.

### TECHNICAL FIELD

The present invention concerns, in general, the technical field of syringes. More particularly, the present invention concerns a safety device that can be applied to any type of commercially available syringe and to a syringe provided with such a safety device.

### BACKGROUND

Contamination by diseases, due to an accidental puncture with a used needle, has serious repercussions not only on the subjects who contract the disease, but also on society, as a result of the associated large health costs. Healthcare professionals in particular are exposed to the risks arising from the use of syringes.

To overcome this drawback, and thus prevent accidental punctures and control in this way the spread of pathogens and infectious agents, syringes are provided with various types of safety devices. Such safety devices include, for example, movable needle covers, which must be manually manipulated to block direct access to the needle tip, and syringes with retractable needle.

The safety devices of the prior art, however, have some drawbacks.

The movable covers, which can be connected to the traditional syringes, are not comfortable and efficient enough, causing in some cases an increase in accidental punctures. The syringes with retractable needle, on the other hand, while efficiently solving the problem of accidental punctures, have high production and sales costs, so they are not very common on the market.

### OBJECTS AND SUMMARY OF THE INVENTION

The main object of the present invention is therefore to make available a safety device for a syringe, capable of overcoming, or at least limiting, the drawbacks associated with the safety devices of known type.

More particularly, the main object of the present invention is to provide a safety device for a syringe configured to hold the needle into position during use or administration of the drug contained in the syringe and allow an automatic unlocking, i.e. without any intervention by a user of the syringe, of the needle and therefore a retraction thereof through the safety device, so that it ends up in the body of the syringe on which it is mounted, after administration. In other words, the user will not be required to intervene with his or her hands to neutralize the syringe needle, thus preventing the operator from accidentally pricking him-or herself, for example when inserting a cap on the needle before disposing of the syringe.

Another object of the present invention is to provide a universal safety device for a syringe, which can be applied, without the aid of adapters, to a traditional pre-filled or to be filled syringe with Luer-slip or Luer-lock fitting.

Yet another object of the present invention is to provide a safety device for a syringe, which is of simple realization and can be produced at competitive costs.

Not the last object of the present invention is to provide a safety device for a syringe, which is simple and of immediate use by a user.

The invention therefore concerns, in a first aspect thereof, a safety device for a syringe comprising a connector which can be coupled to a syringe, a main body provided with a cavity and which can be coupled to the connector so as to extend above it, a needle body for supporting and housing a needle or cannula, wherein the needle body extends longitudinally in the connector and in the main body.

The device further comprises a push element, which can be coupled to a distal end of the needle body and movable inside the connector by action of an axial thrust of a plunger of the syringe, and a needle support which can be coupled to the needle body. The needle support is operable by the push element between a locked position of the needle body, wherein the needle support is interposed between the connector and the needle body, and an unlocked position of the needle body, wherein the needle support is released from the connector and from the needle body and completely housed in the cavity of the main body and the needle or cannula can freely retract through the main body and the connector.

Preferred aspects of the invention are set forth in the dependent claims.

In a second aspect thereof, the invention concerns a syringe provided with a safety device mentioned above.

Thanks to its particular configuration, the safety device for a syringe according to the invention allows to hold into position the needle during use or administration of the drug contained in the syringe and to automatically unlock, i.e. without any intervention of the hands of a user, the needle after administration.

In addition, the universality of the safety device, the geometry completely similar to that of a classic hypodermic needle, hence the development in the longitudinal direction (along the axis of the syringe) and the absence of any type of lateral encumbrance (such as, for example, an encapsulation chamber external to the syringe) are extremely advantageous factors for safely performing an injection and do not require alterations of the technique and/or of the injection angle.

In the following of the present invention, and in the attached claims, the term “proximal” will refer to the portion of a component of the safety device facing the needle or cannula, whereas the term “distal” will refer to the portion of a component of the safety device opposite the needle or cannula, i.e. facing the syringe to which the safety device can be coupled.

### BRIEF DESCRIPTION OF THE DRAWINGS

The invention will be described below with reference to some examples, provided by way of non-limiting example, and illustrated in the appended drawings. These drawings illustrate different aspects and embodiments of the present invention and reference numerals illustrating structures, components, materials and/or similar elements in different drawings are indicated by similar reference numerals, where appropriate.

FIG. 1 is a perspective view of a safety device for a syringe according to a preferred embodiment of the present invention, with the needle covered by a needle cover.

FIG. 2 is a view, similar to that of FIG. 1, with the needle uncovered and ready for use.

FIG. 3 is an exploded view of the safety device of FIG. 1.

FIG. 3A is an enlarged view of the circled detail in FIG. 3, showing the needle support in its locked condition on the needle body.

FIG. 4 is a longitudinal sectional view of the safety device of FIG. 1.

FIG. 5 is a partial and sectional view of the safety device of FIG. 1 mounted on a syringe with Luer-lock fitting, showing the syringe at the time of administration of a product, with the plunger of the syringe approaching the safety device.

FIG. 6 is a partial and sectional view of the safety device of FIG. 1 mounted on a syringe with Luer-lock fitting, showing the plunger of the syringe abutting against the push element of the safety device.

FIG. 7 is a partial and sectional view of the safety device of FIG. 1 mounted on a syringe with Luer-lock fitting, showing the needle support in the unlocked position of the needle body and the needle unlocked and ready to be retracted into the syringe body.

FIG. 8 is a partial and sectional view of the safety device of FIG. 1 mounted on a syringe with Luer-lock fitting, showing the needle now retracted and housed in the syringe body.

### DETAILED DESCRIPTION OF THE INVENTION

While the invention is susceptible to various modifications and alternative constructions, certain preferred embodiments are shown in the drawings and are described hereinbelow in detail. It must in any case be understood that there is no intention to limit the invention to the specific embodiment illustrated, but, on the contrary, the invention intends covering all the modifications, alternative and equivalent constructions that fall within the scope of the invention as defined in the claims.

The use of “for example”, “etc.”, “or” indicates non-exclusive alternatives without limitation, unless otherwise indicated. The use of “includes” means “includes, but not limited to” unless otherwise indicated.

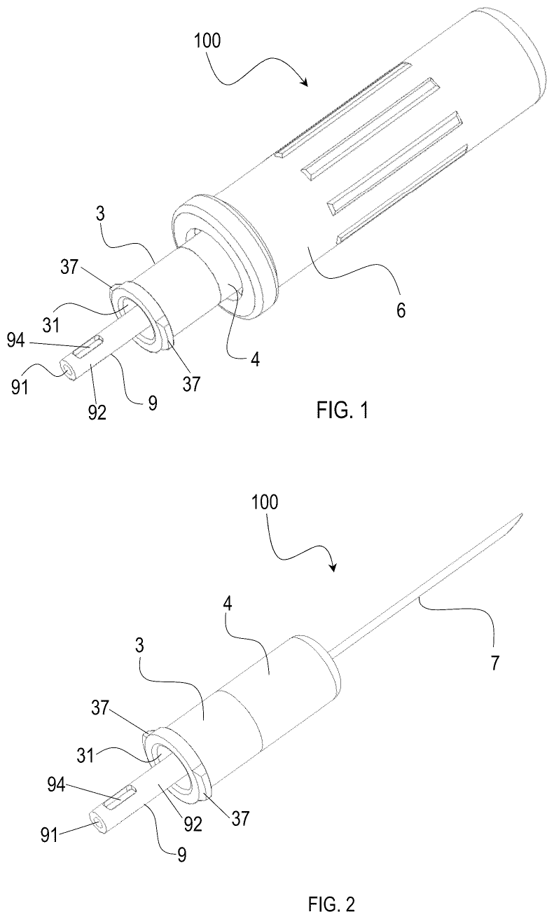

With reference to FIGS. 1 to 4, there is illustrated therein a safety device for a syringe according to a preferred embodiment of the present invention.

The safety device, generally indicated by the reference numeral 100, comprises a connector 3, a main body 4, a needle body 5, a needle or cannula 7, a needle support 8 and a push element 9.

In FIG. 1, the needle or cannula 7 is covered by a needle cover 6, which protects a user, typically a healthcare professional, from needle punctures before using the syringe.

Preferably, the connector 3, the main body 4, the needle body 5, the needle cover 6, the needle support 8 and the push element 9 are made by injection moulding of plastic material, such as for example medical grade polypropylene (PP), polyethylene (PE) or pharmalene. The needle or cannula 7 is typically made of stainless steel.

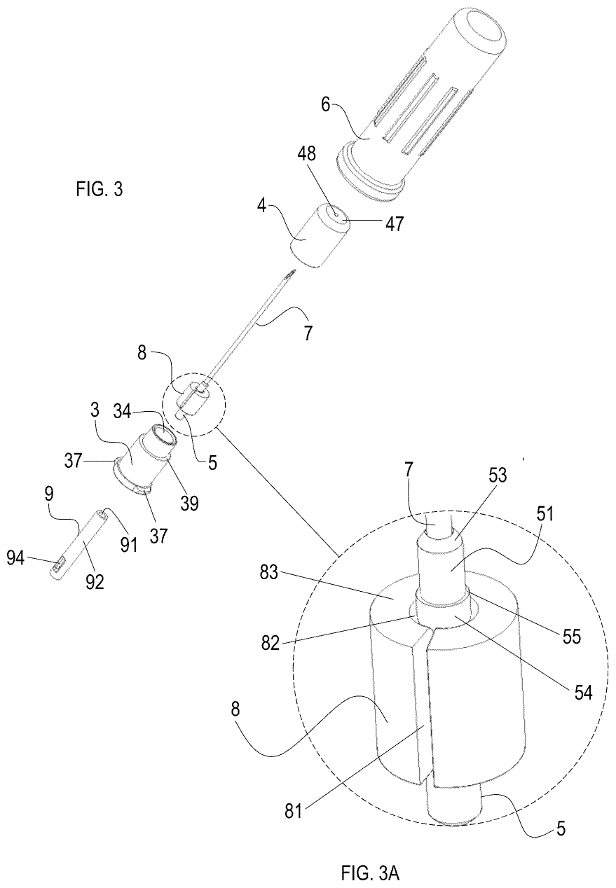

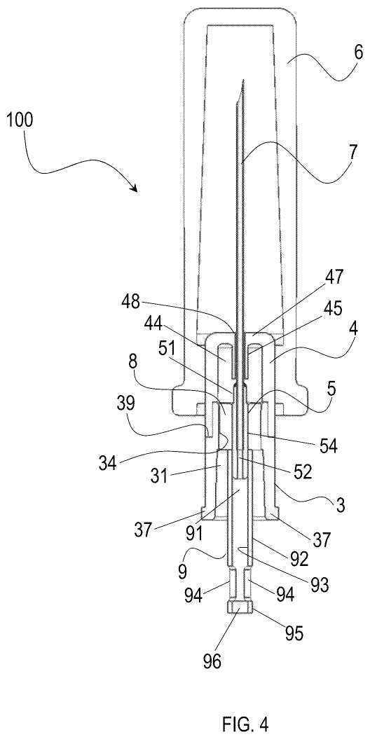

The needle support 8 is configured so that its geometric configuration can be varied during use. In particular, and as shown in detail in FIG. 3A, the needle support 8 has the shape of a hollow cylinder and has a longitudinal slit 81. The presence of such a longitudinal slit 81 allows the needle support 8 to assume two distinct positions, namely a locked position of the needle body 5, shown for example in FIG. 4, and an unlocked position of the needle body 5, shown for example in FIG. 8. Furthermore, in the locked position of the needle body 5, the longitudinal slit 81 allows a greater adhesion of the needle support 8 both to the needle body 5 and to the connector 3, precisely thanks to a greater circumferential elastic deformation of the needle support 8. The needle support 8, instead of being cylindrical, can be frustoconical, with a very small angle at the vertex. The conicity may refer to both the inner surface and to the outer surface of the needle support 8.

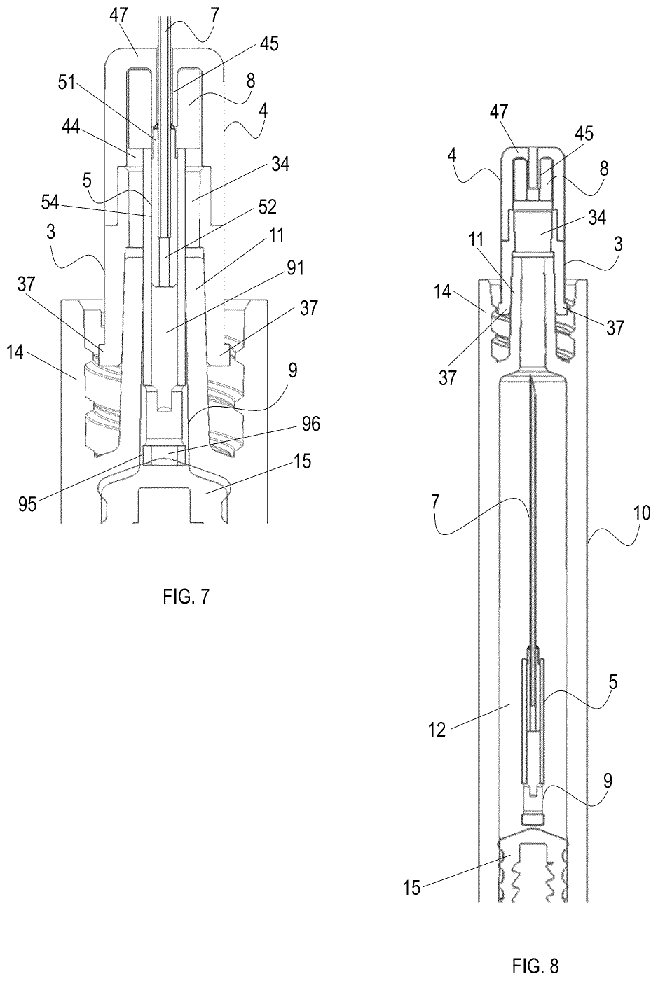

In particular, and as will become clearer in the following of the present description, in the locked position of the needle body 5, the needle support 8 is tightened around the needle body 5 and integral with the connector 3, whereby the needle body 5 is locked in position in the safety device 100. In the unlocked position of the needle body 5, the needle support 8 is integral with the main body 4, with the needle body 5 released from the needle support 8 and therefore free to retract, together with the needle or cannula 7, through the main body 4 and the connector 3, so that it ends its stroke inside the body or barrel 12 of a syringe 10 (see, for example, FIG. 7) on which the safety device 100 is mounted.

The passage from one position to another of the needle support 8 is possible thanks to the application (during mounting of the device) of elastic deformation forces and allows the needle support 8 to perform three main functions: to eliminate any degree of freedom of the needle body 5 in particular during injection, to avoid drug leaks and to unlock the needle body 5 at the end of the injection.

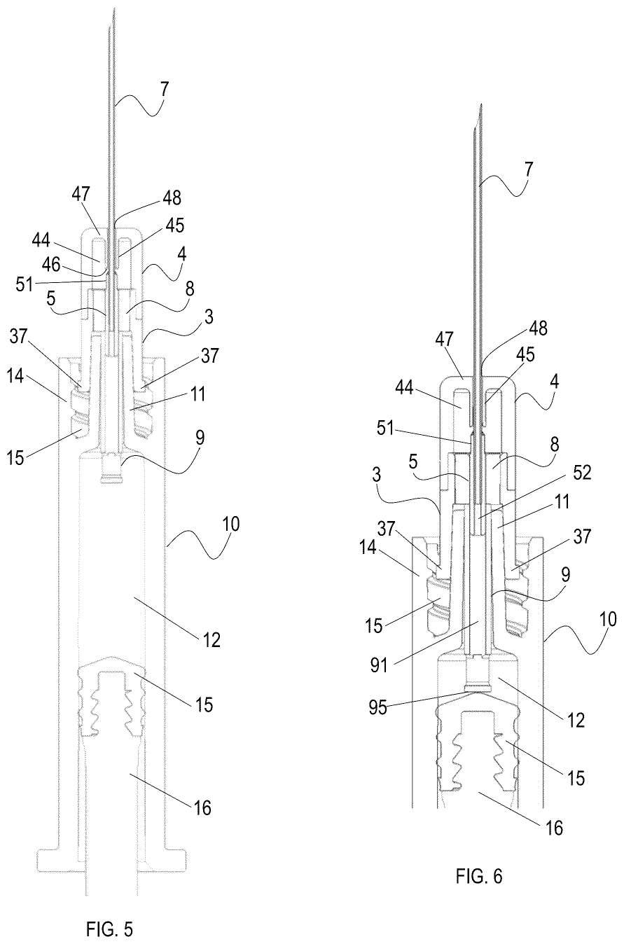

Preferably, the needle support 8 has a chamfer 82 in the proximal end thereof which, as will be better understood hereinafter, facilitates the insertion of the needle support 8 into the main body 4, when the safety device 100 is in operation, mounted on the syringe 10. As shown in detail in FIG. 4, the connector 3 is hollow and has cylindrical side walls, which delimit a first cavity 31, preferably frustoconical, configured for housing a tip 11 (see, for example, FIG. 5) of the syringe 10. Preferably, the connector 3 is further configured for connection of a Luer-lock fitting 14 (see, for example, FIG. 5) of the syringe 10. To this end, two diametrically opposed fins 37, typical of Luer-lock fittings, extend at a distal end of the connector 3.

At a proximal portion of the connector 3 a second cavity 34 develops, also preferably frustoconical, having inverse conicity with respect to that of the first cavity 31. The second cavity 34 is configured to house the needle support 8, closed around a central portion 54 of the needle body 5. The two frustoconical cavities 31 and 34 are into communication and coaxial therebetween.

On the proximal portion of the connector 3 the main body 4 is engaged, which, once engaged, moves into abutment against a shoulder 39 formed in the connector 3.

The main body 4 preferably has a frustoconical shape and presents an outer surface and an inner surface, both frustoconical and with the same inclination, an inner cavity 44, also frustoconical being delimited by the inner surface. The inner cavity 44 has a slightly greater diameter than that of the needle support 8. Furthermore, the main body 4 is closed at the top by a wall 47, which has a through hole 48 for the passage of the needle or cannula 7. In particular, the through hole 48 is dimensioned such that the needle or cannula 7 is free to slide therein.

The main body 4 also has a hollow inner cylinder 45 for housing the needle or cannula 7. In particular, the hollow inner cylinder 45 extends into the inner cavity 44, starting from the upper closing wall 47 of the main body 4, over a length greater than or equal to the height of the needle support 8. The outer diameter of the hollow inner cylinder 45 is slightly greater than the inner diameter of the needle support 8. It follows that, when the needle support 8 is in the unlocked position of the needle body, in the absence of external forces, the needle support is fitted with interference on the hollow inner cylinder 45 of the main body 4, and then locked on the hollow inner cylinder 45 and made integral therewith.

As will be described below, when the needle support 8 is in the above-mentioned unlocked position of the needle body 5, the needle or cannula 7 can freely retract passing through the through hole 48 of the main body 4 and the connector 3. In other words, and as illustrated in FIG. 6, when the needle support 8 is pushed axially, by the push element 9, in turn driven by a plunger 16 of the syringe 10, towards the hollow inner cylinder 45 of the main body 4, the needle support 8 remains connected to the main body 4, tight around the hollow inner cylinder 45, due to the effect of the friction forces developing between the outer surface of the hollow inner cylinder 45 and the inner surface of the needle support 8. The chamfer 82, preferably formed in the needle support 8, facilitates the insertion of the needle body 8 externally to the hollow inner cylinder 45 of the main body 4.

Along the longitudinal axis of the safety device 100 there is placed the needle body 5, preferably cylindrical in shape.

As shown in detail in FIG. 3A, the needle body 5 has, at a proximal end, a portion 51 with narrowing of section, which delimits a shoulder 55. At a head end 53 of the proximal portion 51 with narrowing of section, glue or mastic is preferably applied, which is of aid in keeping the needle or cannula 7 fitted in the needle body 5. The outer diameter of the proximal portion 51 with narrowing of section is smaller than the inner diameter of the needle support 8, whereby any interaction between the outer surface of the proximal portion 51 with proximal narrowing of section of the needle body 5 and the inside of the needle support 8 is avoided. A longitudinal channel 52 (visible in FIG. 4) puts the barrel 12 of the syringe 10 into communication with the needle or cannula 7, with the aim of allowing a product or drug to pass during an injection.

With reference again to FIG. 4, the push element 9 has an outer surface 92 and a longitudinal channel 91. Part of the needle body 5 is housed in a proximal portion of the longitudinal channel 91. In particular, the outer diameter of the needle body 5 is slightly greater than the diameter of the longitudinal channel 91, so as to allow, in the absence of axial forces, an integral and sealed connection between the push element 9 and the needle body 5. Otherwise, i.e. in the presence of an axial force acting on the push element 9, a relative and liquid-tight sliding is allowed between the push element 9 and the needle body 5. In other words, in the absence of a thrust force, the needle body 5 and the push element 9 of the safety device 100 are integral therebetween, but move with respect to each other when an axial force acts on the push element 9 and the needle body 5 were prevented from moving axially.

The push element 9 also has, at a distal end thereof, a transversal through opening 94, which, when the safety device 100 is in use, i.e. mounted on the syringe 10, has the purpose of allowing the passage of the drug from the inside of the syringe up to the needle or cannula 7, from which the drug outflows to be injected into a subject and the passage of air from the inside of the syringe up to the needle or cannula 7, from which the air outflows, immediately before the injection of the drug. Furthermore, in the case of a pre-filled syringe, the transversal through opening 94 of the push element 9 allows the passage, from the inside of the syringe, of the drug contained in the barrel 12 of the syringe 10. Preferably, the transversal through opening 94 has a rectangular section, but may also be circular.

The push element 9 finally has a bottom end 95 provided with a coaxial hole 96, which is nothing more than the extension of the longitudinal channel 91. As will be described in detail below, when the safety device 100 is in use, the bottom surface 95 of the push element 9 serves as an end-of-stroke surface for the plunger 16 of the syringe 10.

When assembling the safety device 100, and as visible in FIG. 4, the needle body 5 is positioned inside the needle support 8, such that the proximal portion 51 with narrowing of section protrudes above from the needle support 8.

The application of axial forces on the proximal upper face 83 causes the insertion of the needle support 8 inside the second frustoconical cavity 34 of the connector 3, whereby the longitudinal slit 81 of the needle support 8 is closed slightly, which facilitates the presence of transversal and circumferential forces that lock the needle body 5, making it actually integral with the needle support 8 and fitted therewith.

Still in the assembly phase, a distal portion of the needle body 5 is housed, with slight interference, in a proximal portion of the longitudinal channel 91 of the push element 9. The housing with interference of the needle body 5 in the push element 9 causes, in the absence of axial forces on the push element 9, the two components to be integral therebetween.

The assembly of connector 3, needle support 8, needle body 5, cannula 7, main body 4 and push element 9 is completed by the insertion of the needle cover 6.

With reference to FIGS. 5 to 8, the operation of the safety device 100 will now be described.

Firstly, a user, typically a healthcare professional, mounts the safety device 100 onto a syringe 10 for administering a drug. If the syringe 10 is of the pre-filled type, the drug is already present in the barrel 12 of the syringe. Otherwise, the drug should be aspirated from a vial (not shown) containing it. To this end, the connector 3 is coupled with interference on a tip 11 of the syringe 10. In the case of a syringe with Luer-lock fitting, in this coupled position, the fins 37 of the connector 3 are engaged with a thread of the inner threading 15 of the Luer-lock 14.

Thereafter, the needle cover 6 is removed, so as to leave the needle or cannula 7 uncovered. The mounted condition of the safety device 100 is shown, for example, in FIG. 5. Furthermore, and as visible in FIG. 5, when the safety device 100 is mounted on the tip 11 of the syringe 10, the distal portion of the push element 9 extends into the body or barrel 12 of the syringe 10. This operating condition is shown in FIG. 5.

In this operating configuration, the needle or cannula 7 is fixed, integral with the main body 4 and with the connector 3. In case of axial forces applied on the needle or cannula 7, for example while inserting the needle into the skin and tissues of a patient, the needle or cannula 7, by pushing on the inner walls of the needle support 8, pushes the latter to further fit, due to the effect of the conicity of the inner surface of the needle support 8 and to the conicity of the outer surface of the central portion 54 of the needle body 5, inside the second cavity 34 of the connector, and therefore to make the needle body 5 and consequently the needle or cannula 7, even tightened inside the safety device 100. This advantageously increases the reliability of the device 100, just when it is mostly required, i.e. at the time of the injection of the drug. It follows that, both the patient and the nurse performing the injection do not perceive any difference with respect to the syringes normally available on the market.

Once the needle cover 6 is removed, the user presses the plunger 16 of the syringe, until a rubber 15, mounted on the head of the plunger 16, comes into contact with the bottom end 95 of the push element 9 of the safety device 100. This operating condition is shown in FIG. 6.

In case instead of using a non-pre-filled type syringe, the needle or cannula 7 is inserted into the vial, with the aim of taking the drug contained therein. After inserting the needle or cannula 7 into the vial, the plunger 16 is retracted allowing the drug to enter the barrel 12, by passing through a longitudinal hole of the needle or cannula 7 and subsequently through the longitudinal channels 52, 91 and the hole 96, respectively, of the needle body 5 and the push element 9. This optional operating step is not illustrated in the figures.

Before injection, the escape of any air present in the barrel 12 is facilitated by the transversal through opening 94 and by the hole 96 formed, respectively, in the distal end and in the bottom end 95 of the push element 9. The advancement of the plunger 16 inside the barrel 12 in fact pushes the air through the opening 94, therefore through the push element 9 and the needle or cannula 7.

Then the drug is injected. In this step, the operating configuration of the needle support 8 prevents the needle or cannula 7 from moving along the axis of the syringe 10 during perforation of the dermis and hinders unwanted drug leaks during injection. The drug that is not channelled into the needle body 5, but dispersed within the connector 3, is in fact blocked at the level of the needle support 8.

At the end of the injection, the rubber 15 of the plunger 16 of the syringe 10 is in abutment against the bottom end 95 of the push element 9. This operating condition is shown in FIG. 6.

At this point, and as visible in FIG. 7, the further advancement of the plunger 16, and of the relative rubber 15, in addition to allowing to inject any residual drug still present in the barrel 12, axially pushes the push element 9, with consequent upward advancement of the needle body 5 integral thereto, until when the head end 53 of the needle body 5 moves into mechanical abutment against an annular lower surface of the hollow inner cylinder 45. When this happens, a further stroke of the piston 16 causes a further advancement of the push element 9, which, in turn, advances the needle support 8, which is released from the needle body 5, around which it is mounted, moving into its unlocked position of the needle body 5.

In such an unlocked position of the needle body 5, the needle support 8 leaves the second cavity 34 of the connector 3 free to be housed in the cavity 44 of the main body 4, locked on the hollow inner cylinder 45, and made integral therewith. An interval of forces between about 1.5 N and about 2.0 N is sufficient to make the plunger 16 reach the end of stroke.

In such a configuration, the needle support 8, no longer subjected to the contact forces with the connector 3, is integral with the main body 4, as it is held still by the static friction forces developing due to the pressing force that the needle support 8 exerts on the outer surface of the hollow inner cylinder 45.

In this regard, it should be noted that the further advancement of the plunger 16, with consequent unlocking of the needle or cannula 7, entails an imperceptible displacement, typically of the order of a few tenths of a millimetre, of the needle or cannula 7 within the patient's body. Such imperceptible displacement stops when the head end 53 of the needle body 5 moves into mechanical abutment against an annular lower surface of the hollow inner cylinder 45.

Upon ending the injection, the needle or cannula 7 is extracted from the patient's body. During the extraction of the needle or cannula 7 from the dermis, an edge 46, visible in FIG. 5, of the hollow inner cylinder 45 of the main body 4 acts as a directional mechanical constraint for the needle body 5, allowing the needle or cannula 7 not to remain in the dermis of the patient and therefore to overcome the friction provided by the dermis.

Once the needle or cannula 7 is extracted from the dermis, and by holding the syringe 10 into an upright position, with the tip 11 facing upwards, the plunger 16 and the rubber 15 are made to slide backwards into the barrel 12. The retraction of the plunger 16 causes the retraction of the needle or cannula 7, together with the needle body 5 and with the push element 9 to which the needle body 5 is connected by friction, through the main body 4 and the connector 3 of the safety device 100 and their subsequent fall by gravity, inside the barrel 12 of the syringe 10, with consequent encapsulation of the needle or cannula 7 inside the barrel 12. Encapsulation of the needle or cannula 7 prevents any accidental puncture of the operator. This operating condition is shown in FIG. 8.

It should be also noted that small defects such as burrs, or slight geometric imperfections of the components of the safety device, do not negatively impact on the retraction of needle 7, needle body 5 and push element 9 inside the barrel 12 of the syringe 10.

Attaching the needle cover 6 seals the safety device 100 and makes the syringe 10-safety device 100 assembly ready for disposal.

From the above description the features of the safety device object of the present invention, as well as the advantages thereof, are evident. The safety device is simple to use and universal and applicable to any commercial syringe with a Luer-slip or Luer-lock tip. The costs of realization and assembly are very low. The few constituent elements are in fact easy to realize and provide for mounting along a single axis.

Finally, it is clear that the safety device thus conceived is susceptible of numerous modifications and variations; moreover, all the details can be replaced by technically equivalent elements. In practice, the materials used, as well as their dimensions, can be of any type according to the technical requirements.

1-11. (canceled)

12. A safety device for a syringe comprising:
a connector configured to be coupled to a syringe;
a main body provided with a cavity and configured to be coupled to the connector so as to extend above it; and
a needle body for supporting and housing a needle or cannula, the needle body longitudinally extending in the connector and in the main body;
wherein the device further comprises a push element for coupling a distal end of the needle body and movable inside the connector by action of an axial thrust of a plunger of the syringe, and a needle support configured to be coupled to the needle body and operable by the push element between a locked position of the needle body, wherein the needle support is interposed between the connector and the needle body, and an unlocked position of the needle body, wherein the needle support is released from the connector and from the needle body and completely housed in the cavity of the main body and the needle or cannula can freely retract through the main body and the connector.

13. The safety device according to claim 12, wherein the connector has a first cavity for housing a tip of the syringe and a second cavity, for housing at least one portion of the needle support, the first and second cavities being into communication and coaxial therebetween.

14. The safety device according to claim 13, wherein the first cavity and the second cavity are frustoconical and have inverse conicity.

15. The safety device according to claim 12, wherein the main body is closed at the top by a wall, from which a hollow inner cylinder extends into the cavity of the main body, wherein the wall has a hole for the passage of the needle or cannula.

16. The safety device according to claim 15, wherein the needle support has a substantially frustoconical shape and presents an outer surface and an inner surface, both frustoconical, and a longitudinal slit, wherein an inner diameter of the needle support is smaller than an outer diameter of the hollow inner cylinder, so that when the needle support is coupled to the hollow inner cylinder, the needle support is held into position inside the main body, due to the effect of a static friction force which is generated between the needle support and the hollow inner cylinder.

17. The safety device according to claim 12, wherein the push element has a longitudinal channel for coupling the distal end of the needle body, wherein the longitudinal channel has a diameter smaller than an outer diameter of the needle body, so as to allow, in the absence of axial forces, an integral and sealed connection between the push element and the needle body.

18. The safety device according to claim 17, wherein the push element further comprises, at a distal end thereof, a transversal through opening and a bottom end in which a hole for extending the longitudinal channel is formed, for the passage of drug or air.

19. The safety device according to claim 12, wherein the needle body has a longitudinal channel and a proximal portion with narrowing of section, so as to delimit a shoulder.

20. The safety device according to claim 12, wherein, in the locked position of the needle support, the needle body, the needle or cannula and the push element are integral and fixed therebetween in all phases of use of the device.

21. The safety device according to claim 12, wherein the needle body has a central portion for coupling the needle support in its locked position, which central portion (has a conical outer surface.

22. A syringe comprising a tip, a body and a plunger movable longitudinally in the body, and further comprising a safety device according to claim 12, wherein when the needle support is in the unlocked position of the needle body, the needle body and the push element to which it is integral, fall inside the body of the syringe.
